(19) United States
(12) Plant Patent Application Publication
(10) Pub. No.: US 2016/0029536 P1
(43) Pub. Date: Jan. 28, 2016

(54) Calibrachoa plant named 'KLECA13248'

(50) ;

(71) Applicant: Klemm+Sohn GmbH & Co., KG, Stuttgart (DE)

(72) Inventors: Nils Klemm, Stuttgart (DE); Anita Stöver, Stuttgart (DE)

(73) Assignee: Klemm+Sohn GmbH & Co., KG, Stuttgart (DE)

(21) Appl. No.: 14/121,074

(22) Filed: Jul. 28, 2014

(51) Int. Cl. A01H 5/00

(52) U.S. Cl. PLT/413

(57) ABSTRACT

A new Calibrachoa plant particularly distinguished by having a red-purple flower color and a semi-trailing plant habit, is disclosed.

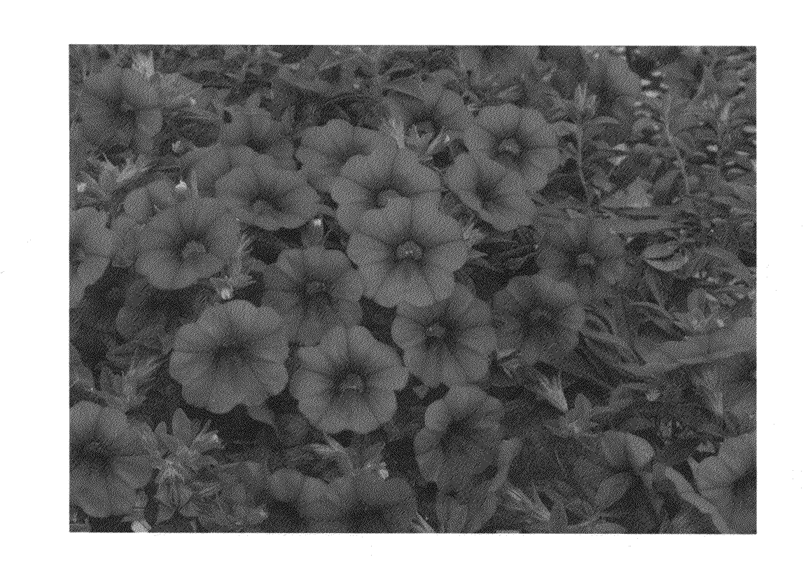

### GENUS AND SPECIES

Calibrachoa sp.

### VARIETY DENOMINATION

‘KLECA 13248’

### BACKGROUND OF THE NEW PLANT

The present invention comprises a new and distinct variety of Calibrachoa, botanically known as Calibrachoa sp., and hereinafter referred to by the variety name ‘KLECA 13248’. ‘KLECA 13248’ originated from a controlled-cross between the female Calibrachoa variety ‘KLECA08170’ (U.S. Plant Pat. No. 21,061) and the proprietary male Calibrachoa variety ‘CA-2008-0300’ (unpatented).

In May 2009, two Calibrachoa parental lines, the female variety ‘KLECA08170’ and the male Calibrachoa variety ‘CA-2008-0300’ were crossed via a controlled cross in Stuttgart, Germany and seeds were obtained. The seeds were sown and plants were grown for evaluation. A plant line was selected in May 2010 which had a red-purple flower color and subsequently named ‘KLECA13248’. In May 2010, ‘KLECA13248’ was first vegetatively propagated by tip cuttings in Stuttgart, Germany. ‘KLECA13248’ was found to reproduce true to type in successive generations of asexual propagation via tip cuttings.

### SUMMARY

The following are the most outstanding and distinguishing characteristics of this new variety when grown under normal horticultural practices in Stuttgart, Germany.

1. Red-purple flower color; and

2. A semi-trailing plant habit.

### DESCRIPTION OF THE PHOTOGRAPH

This new Calibrachoa plant is illustrated by the accompanying photograph which shows a close-up of the flowers. The photograph is of a five-month-old plant grown in a controlled greenhouse environment in Stuttgart, Germany in May 2012. The colors shown are as true as can be reasonably obtained by conventional photographic procedures.

### DESCRIPTION OF THE NEW VARIETY

The following detailed descriptions set forth the distinctive characteristics of ‘KLECA13248’. The data which define these characteristics were collected from asexual reproductions carried out in Stuttgart, Germany. Data was collected on eleven-weeks-old plants grown in Stuttgart, Germany June 2014. Color references are to The R.H.S. Colour Chart of The Royal Horticultural Society of London (R.H.S.), 5^{th} edition (2007).
Classification:
   Family.—Solanaceae.
   Species.—Calibrachoa sp.
   Common name.—Calibrachoa.
   Parentage.—Female: Proprietary female Calibrachoa ‘KLECA081710’ Male: Proprietary male Calibrachoa ‘CA-2008-0300’ (unpatented).
Plant description:
   Life cycle.—Annual.
   Form.—Semi-trailing.
   Habit.—Semi-trailing.
   Height (from soil line to top of foliage).—5.0 cm.
   Spread.—30.0 cm.
Propagation: Vegetatively propagated
   Type cuttings.—Tip cuttings.
   Time to produce a rooted cutting.—19 days.
Stems:
   Stem color.—RHS 146A.
   Pubescence.—Present.
   Pubescence color.—RHS 146C.
   Stem diameter.—0.3 cm.
   Stem length.—15.0 cm From soil line to first node: 1.3 cm Total length: 16.3 cm.
   Internode length.—2.0 cm.
Leaves:
   Arrangement.—Alternate, simple, sessile.
   Shape.—Broad elliptic.
   Apex.—Obtuse to slightly acute.
   Base.—Attenuate.
   Margin.—Entire.
   Surface.—Smooth.
   Surface pubescence.—Absent.
   Venation.—Pinnate.
   Venation color.—Upper surface: RHS 138A Lower surface: RHS 138C.
   Length.—2.7 cm.
   Width.—1.2 cm.
   Color.—Upper surface: RHS N137A Lower surface: RHS 138B.
   Fragrance.—Absent.
Flowers: Single flowers
   Flowering habit.—Flower attitude semi-erect, single, axillary.
   Flower type.—Determinate inflorescence.
   Flowering requirements.—A minimum of light sun is required.
   Duration of flowers.—6 days.
   Corolla.—Medium texture.
   Shape.—Trumpet shaped.
   Fragrance.—Absent.
Flower buds:
   Surface.—Smooth.
   Length.—2.0 cm.
   Diameter.—0.6 cm.
   Shape.—Irregular to oblong.
   Color.—RHS N81A.
Peduncle:
   Length.—1.2 cm.
   Diameter.—0.1 cm.
   Color.—RHS 144B.
   Texture.—Smooth.
   Anthocyanin color.—RHS N77C.
Flower description:
   Flower depth.—2.8 cm.
   Flower tube length.—1.6 cm.
   Flower tube diameter.—1.1 cm.
   Flower diameter.—3.6 cm.
   Pedicel color.—RHS 144B.
   Calyx.—Funnel-shaped.
Sepals:
   Shape.—Lanceolate.
   Apex.—Broad acute.
   Margin.—Entire.
   Length.—1.5 cm.
   Diameter.—0.4 cm.
   Sepal color.—Upper surface: RHS 144A Lower surface: RHS 146B
Petals:
   Shape.13 Heart-shaped.
   Length.—1.9 cm.
   Apex.—Obverse.
   Margin.—Entire, weak undulation.
   Base.—Open and fused in the tube.
   Pubescence.—Absent.
   Lobe length.—1.8 cm.
   Lobe width.—1.8 cm.
   Color.—Lobe color: Upper surface: RHS 67A Lower surface: RHS 67B Corolla tube color: Inner: RHS N77A Outer: RHS N77A.
   Fragrance.—Absent.
Reproductive organs:
   Stamen number.—5.
   Stamen color.—Anther color: RHS 7B Filament color: RHS 10B Pollen color: RHS 7B
   Ovary.—Placenta arrangement: Ovate Pistil number: 1 Pistil length: 0.1 cm Stigma color: RHS 154A Style length: 0.9 cm Style color: RHS 149B.
   Fruit/seed set.—Small.
Disease/insect resistance: None observed

### COMPARISON WITH PARENTAL LINES AND KNOWN VARIETY

‘KLECA13248’ is distinguished from its female parent as described in Table 1:

TABLE 1
Comparison with Parental Lines

| | Variety | Female parent | Male Parent |
|---|---|---|---|
| Trait | ‘KLECA13248’ | ‘KLECA08170’ | ‘CA-2008-0300’ |
| Flower color | Red-purple | Light blue | Red |

‘KLECA13248’ is most similar to the Calibrachoa plant named ‘KLECA10218’ (U.S. Plant Pat. No. 22,892). Differences between the two varieties are described in Table 2:

TABLE 2
Comparison with Similar Variety

| | Variety | Commercial line |
|---|---|---|
| Trait | ‘KLECA13248’ | ‘KLECA10218’ |
| Habit | Semi-trailing | Semi-upright |

We claim:

1. A new and distinct variety of Calibrachoa plant named ‘KLECA13248’ as shown and described herein.
